(19) United States
(12) Patent Application Publication
(10) Pub. No.: US 2022/0327649 A1
(43) Pub. Date: Oct. 13, 2022

(54) SYSTEM AND METHOD FOR MANAGING SOCIAL NEEDS OF A CLIENT

(71) Applicants: Michael Rocky Dotson, Houston, TX (US); Palak Rajesh Jalan, Houston, TX (US)

(72) Inventors: Michael Rocky Dotson, Houston, TX (US); Palak Rajesh Jalan, Houston, TX (US)

(21) Appl. No.: 17/228,894

(22) Filed: Apr. 13, 2021

(51) Int. Cl. G06Q 50/22; G16H 10/60; G06F 16/955

(52) U.S. Cl. CPC G06Q 50/22; G16H 10/60; G06F 16/9554

(57) ABSTRACT

A system is provided for managing needs of a client. The system includes at least one processor configured to obtain client data of a client and predict at least one need of the client based at least on the client data. The at least one processor is further configured to obtain information relating to a plurality of service providers, wherein each service provider provides at least one service relating to a corresponding need. The at least one processor is configured to provide personalized referral of a service provider to the client, based at least on the client data and the predicted at least one need of the client.

### TECHNICAL FIELD

The present disclosure relates generally to managing social needs of a client, and more specifically to intelligent prediction of social needs of a client, automatic referral of service providers to service the social needs and post referral tracking of service fulfillment.

### BACKGROUND

Health care providers such as hospitals often have processes set up to help patients with their social needs such as food, housing, clothing, education, employment, recreation and the like. However, determining social needs of the patients and providing them referrals to service providers to service their needs requires personal consultation with a care coordinator given that the health care provider has the necessary resources to make care coordinators available to patients. During such a consultation, the care coordinator determines social needs of the patient by asking pointed questions and identifies service providers the patient qualifies for and which also meet one or more patent requirements such as proximity to patient's residence, hours of operation, languages spoken and the like. The need for person to person contact with care coordinators to provide care to patients relating to their social needs places undue burden on health care organizations. Further, needing direct contact with a care coordinator is often a bottle neck in providing care to the patients in a timely manner. For example, studies have shown that one care coordinator is needed to service about 175 patients. This means a health care organization having tens of thousands of patients needs to employ hundreds of care coordinators to service all patients. This places a considerable financial and processing burden on the organization. Currently, there is no easier and cheaper way to provide personalized care to patients with regard to their social needs.

### SUMMARY

The system and methods implemented by the system as disclosed in the present disclosure provide technical solutions to the technical problems discussed above by intelligently predicting client needs and providing personalized referrals of service providers to service the client needs. The disclosed system and methods provide several practical applications and technical advantages. In one or more embodiments, the disclosed system predicts one or more needs of a client by identifying patterns in client data relating to a plurality of clients. For example, the system identifies correlation patterns in the client data relating to a plurality of clients, wherein each correlation pattern correlates or maps one or more client data points with a client need. In order to identify a correlation pattern in the client data, the system correlates client data points relating to a plurality of clients who have at least one common known need, and identifies common values of data points between the plurality of clients. The system predicts one or more client needs based on the identified correlation patterns. The disclosed system also provides personalized referrals of one or service providers to clients for servicing one or more client needs. For example, the system searches a service provider database for service providers that can service needs of a client and provides one or more service providers as referrals for the client. The system may also cross reference the client data of the client with the service provider data stored in the service provider database based on one or more client requirements to provide service provider referrals which meet the client requirements. Once the service provider referrals are determined, the system automatically sends out personalized notifications to the client including information relating to the referred service providers and other instructions to receive services from the service providers. Thus, the system and methods described in this disclosure considerably reduce, and in some cases, eliminate the need for a patient to have direct contact with a care coordinator for receiving care related to social needs of the patient. This reduces the financial and processing burden on health care organizations and removes the bottleneck in servicing patients associated with direct contact with care coordinators.

Certain aspects of the present disclosure may include some, all, or none of these advantages. These advantages and other features will be more clearly understood from the following detailed description taken in conjunction with the accompanying drawings and claims.

In certain aspects, a system includes at least a triage entity and a service provider entity. The triage entity is configured to obtain client data of a client and predict at least one need of the client based at least on the client data. The triage entity obtains information relating to a plurality of service providers, wherein each service provider provides at least one service relating to a corresponding need. The triage entity provides personalized referral of a service provider to the client, based at least on the client data and the predicted at least one need of the client. The service provider entity is associated to the service provider and is configured to receive information related to the referral, wherein the information related to the referral comprises one or more of at least a portion of the client data of the client, information relating to the need of the client and copies of one or more documents needed to establish that the client satisfies a registration criterion of the service provider. A scanner coupled to the service provider entity is configured to scan a machine-readable code provided by the client, wherein the service provider entity transmits information related to the scan to the triage entity, wherein the information related to the scan indicates that the client has received services by the service provider.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 is a schematic diagram of a system for managing social needs of clients, in accordance with certain embodiments of the present disclosure;

FIG. 2A and FIG. 2B show an example email notification sent out to a client, in accordance with certain embodiments of the present disclosure;

FIG. 3 is a flowchart of an example method for managing social needs of a client, in accordance with certain embodiments of the present disclosure; and

FIG. 4 illustrates an example schematic diagram of the triage entity, in accordance with one or more embodiments of the present disclosure.

### DETAILED DESCRIPTION

### System Overview

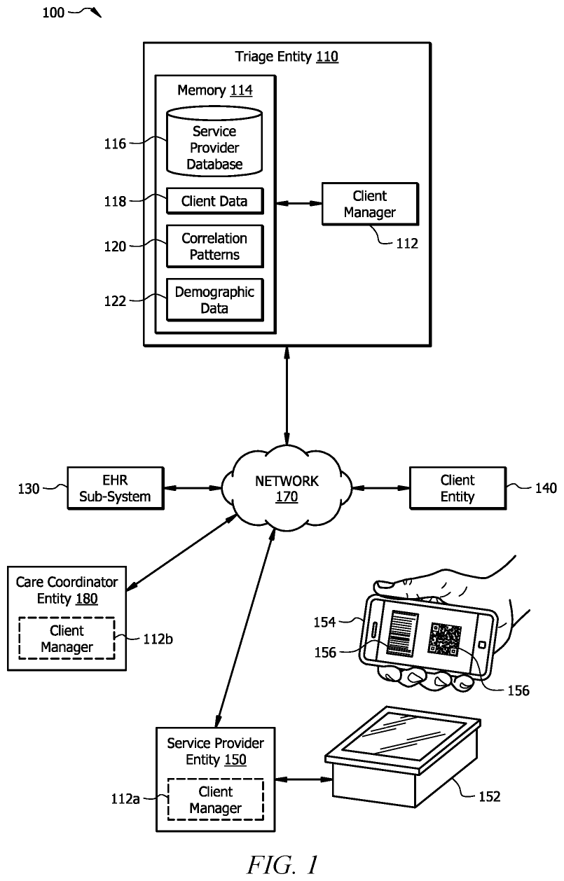

FIG. 1 is a schematic diagram of a system 100 for managing social needs of clients, in accordance with certain embodiments of the present disclosure.

It may be noted that the term “client” as used with reference to certain embodiments of this disclosure refers to a person, such as a patient at a health care facility which may use system 100 to identify needs of the patient, provide personalized referrals of service providers to the patient and track fulfillment of services at the referred service providers.

As shown in FIG. 1, system 100 includes a triage entity 110, Electronic Health Records (EHR) sub-system 130, client entity 140, service provider entity 150 and care coordinator entity 180, each connected to a network 170. The network 170, in general, may be a wide area network (WAN), personal area network (PAN), a cellular network, or any other technology that allows devices to communicate electronically with other devices. In one or more embodiments, the network 170 may be the internet.

In one or more embodiments, each of the triage entity 110, Electronic Health Records (EHR) sub-system 130, client entity 140, service provider entity 150 and care coordinator entity 180 may be implemented by a computing device running one or more software applications. For example, one or more of the triage entity 110, Electronic Health Records (EHR) sub-system 130, client entity 140, service provider entity 150 and care coordinator entity 180 may be representative of a computing system hosting software applications that may be installed and run locally or may be used to access software applications running on a server (not shown). The computing system may include mobile computing systems including smart phones, tablet computers, laptop computers, or any other mobile computing devices or systems capable of running software applications and communicating with other devices. The computing system may also include non-mobile computing devices such as desktop computers or other non-mobile computing devices capable of running software applications and communicating with other devices. In certain embodiments, one or more of the triage entity 110, Electronic Health Records (EHR) sub-system 130, client entity 140, service provider entity 150 and care coordinator entity 180 may be representative of a server running one or more software applications to implement respective functionality as described below. In certain embodiments, one or more of the triage entity 110, Electronic Health Records (EHR) sub-system 130, client entity 140, service provider entity 150 and care coordinator entity 180 may run a thin client software application where the processing is directed by the thin client but largely performed by a central entity such as a server (not shown).

### Triage Entity

The triage entity 110 may be configured to perform one or more functions including predicting client needs, providing personalized referrals of service providers to clients and tracking fulfillment of services at the service providers. In one or more embodiments, the triage entity 110 may belong to any provider of triage services including health care providers such as hospitals and other medical facilities. It may be noted that while embodiments of the present disclosure are described with reference to the triage entity 110 implemented and used by a health care provider such as a hospital or any other medical facility, a person of ordinary skill in the art can appreciate that the triage entity 110 may be implemented and used in the manner described in this disclosure by any individual or organization having access to client data and service provider data. In this context, the terms “client” and “patient” are used interchangeably throughout this disclosure.

In the context of the present disclosure, a service provider may include any individual or organization that provides social assistance to clients with regard to one or more client needs. Client needs may include, but are not limited to, assistance with food, housing, clothing, utilities, transportation, legal needs, exercise and recreational facilities, employment and education. By way of example, a food bank may be one such service provider that helps clients with food insecurities. It may be noted that a single service provider may provide multiple services to service several social needs of clients. A service provided by a service provider may include, but are not limited to, assistance with food, housing, clothing, utilities, transportation, legal needs, exercise and recreational facilities, employment and education.

As shown in FIG. 1, the triage entity 110 includes client manager 112 and memory 114. In an embodiment, client manager 112 is a software application that is run by a local processor at the triage entity 110 based on software instructions stored in memory 114. In an alternative embodiment, the client manager 112 may be a thin client software application where the processing is directed by the thin client but largely performed by a server (not shown). The memory 114 stores a service provider database 116, client data 118, correlation patterns 120 and demographic data 122. Client data 118 may include electronic health records (EHR) of clients (e.g., patients) and other information provided by clients to the health care provider managing the triage entity 110. An EHR generally refers to a collection of electronically stored health information of a patient in digital format. EHRs may include a range of data including, but not limited to, demographics, medical history, medication and allergies, immunization status, laboratory test results, radiology images, vital signs, personal statistics like age and weight, and billing information. Currently, there are over 500 EHR vendors offering some form of EHR product. In one or more embodiments, the triage entity 110 may obtain EHR data of clients from EHR sub-system 130. EHR sub-system 130 may belong to any commercial EHR vendor providing EHR data. In one or more embodiments, the EHR sub-system 130 may include several EHR sub-systems, wherein each of the several EHR sub-systems is managed by a different EHR vendor. The triage entity 110 may be configured to obtain EHR data from EHR sub-systems 130 of one or more EHR vendors. The triage entity may be configured to receive EHR data from the EHR sub-system 130 in a standard file format such as in a Comma-separated values (CSV) file. The triage entity 110 may be configured to receive EHR data from the EHR sub-system 130 periodically and/or according to a pre-determined schedule.

Client data 118 may also include information provided by patients to a health care provider. For example, a patient, upon a visit to a hospital, may be asked to fill out a questionnaire asking the patient to provide information including, but not limited to, age, gender, date of birth, social security number, income, home address, work address, weight, education level, employment status, military status, living situation and insurance status.

Service provider database 116 may include information relating to a plurality of service providers providing an array of services to meet client needs. For example, information relating to a service provider may include several data points including, but is not limited to, legal name, tax identification (ID), general point of contact, full address (including city, state and zip code), office phone number, mobile phone number, email address, service location(s), service(s) provided and hours of operation, languages spoken, total capacity to handle clients, capacity for individual services, one or more registration criterion, documents required (e.g., insurance, proof of ID, address, military status etc.).

In one or more embodiments, client manager 112 is configured to predict one or more needs of a client by identifying patterns in client data 118 relating to a plurality of clients. In one embodiment, client manager 112 is configured to identify correlation patterns 120 in client data, wherein each correlation pattern 120 correlates or maps one or more client data points with a client need. For example, a correlation pattern 120 may indicate that 90% of clients living in a particular zip code and having a household income less than $30,000 a year are food insecure. Following this example, for every client who lives in the same particular zip code and has a household income less than $30,000, client manager 112 may predict that the client is food insecure based on the correlation pattern 120.

Additionally or alternatively, client manager 112 may be configured to identify correlation patterns 120 in client data 118 based on demographic data 122 in order to predict client needs of clients. Such correlation patterns 120 may be referred to as demographic correlation patterns. For example, client manager 112 may have previously identified a data pattern based on client data 118 that 90% of clients living in a particular zip code have a household income less than $30,000 a year and are food insecure. Demographic data 122 may include data relating to other zip codes in the same city which have more than 90% of people living with household incomes less than $30,000 a year. Based on the previously identified data pattern in client data 118 and demographic data 122, client manager 112 may determine a demographic correlation pattern 120 which indicates that clients living in any zip code within the city and having household incomes lower than $30,000 a year are likely to be food insecure. Following this example, for every client who lives in any zip code of the city and has a household income less than $30,000, client manager 112 may predict that the client is food insecure based on the demographic correlation pattern 120.

In one or more embodiments, in order to identify a correlation pattern 120 in the client data 118, client manager 112 correlates client data points relating to a plurality of clients who have at least one common known need, and identifies common values of data points between the plurality of clients. For example, client manager 112 compares client data points of a plurality of clients known to be food insecure and identifies a subset of clients from the plurality of clients who live in the same zip code and have household incomes less than $30,000. In one embodiment, client manager 112 may designate an identified pattern in client data 118 as a correlation pattern 120 when more than a threshold number of clients having the same known need are identified to have the same one or more common data point values. For example, when client manager 112 identifies that more than 80% of clients living in the same particular zip code and having household incomes less than $30,000 are food insecure, client manager 112 designates this pattern as a correlation pattern 120 and stores the identified correlation pattern 120 in memory 114.

Once the correlation patterns 120 are identified and stored in memory 114, for every client, client manager 112 may correlate client data 118 of the client with each correlation pattern 120 stored in memory 114. When one or more data point values of the client data 118 of the client matches with corresponding one or more data point values of a correlation pattern 120, client manager 112 predicts that the client has a need associated with the correlation pattern 120. For example, when a correlation pattern 120 indicates that any client living in a particular zip code and having household income less than $30,000 is food insecure, any client living in the same particular zip code and having household incomes less than $30,000 is predicted to be food insecure.

In one or more embodiments, in order to identify a demographic correlation pattern 120, client manager 112 may correlate client data points in client data 118 relating to a plurality of clients who have at least one common known need, as well as to demographic data 122. Client manager 112 identifies common values of data points between the plurality of clients and compares the identified common data point values with demographic data 122, in order to identify demographic correlation patterns.

For example, client manager 112 may identify based on client data 118 that 90% of clients living in a particular zip code and having a household income less than $30,000 a year and are food insecure. Client manager 112 compares this correlation with demographic data 122 which may include data relating to other zip codes in the same city which have more than 90% of people with household incomes less than $30,000 a year. Based on the comparison of the previously identified correlation of the client data point values in the client data 118 and additionally demographic data 122, client manager 112 may determine a demographic correlation pattern 120 which indicates that clients living in any zip code within the city having household incomes lower than $30,000 a year are likely to be food insecure.

Following this example, one the demographic correlation pattern 120 is identified and stored in memory 114, for every client, client manager 112 may correlate client data 118 of the client with the demographic correlation pattern 120 stored in memory 114. When one or more data point values of the client data 118 of the client matches with corresponding one or more data point values of the demographic correlation pattern 120, client manager 112 predicts that the client has a need associated with the demographic correlation pattern 120. For example, for every client who lives in any zip code of the city and has a household income less than $30,000, client manager 112 may predict that the client is food insecure based on the demographic correlation pattern 120.

In one or more embodiments, triage entity 110 may be run in a training mode to help collect client data 118 and identify correlation patterns 120, before the client manager 114 can make reasonably accurate predictions of client needs based on the identified correlation patterns. When the triage entity 110 is running in the training mode, clients may be asked to self-screen (e.g., explicitly state) their needs. Client manager 112 correlates client data 118 relating to a plurality of clients who self-screened as having a common need and identifies correlation patterns as described above based on the correlations. In an embodiment, triage entity 110 may be run in the training mode for a pre-determined time period or till sufficient client data is collected to identify correlation patterns for making reasonably accurate predictions of client needs.

In one or more embodiments, a health care provider may randomly, periodically or based on a pre-determined schedule ask patients to explicitly state their needs. The stated needs of the patients may be manually entered using a user interface (not shown) of the triage entity 110. The client manager 112 is configured to check predictions of social needs of one or more clients against the explicitly stated needs of the same clients in order to determine an accuracy of predictions. The client manager 112 may be configured to re-evaluate one or more correlation patterns 120 when the accuracy of predictions made based on the correlation patterns 120 falls below a threshold accuracy. Re-evaluating a correlation pattern 120 may include re-running the correlation associated with the correlation pattern 120 based a current set of client data 118.

In one or more embodiments, client manager 112 is configured to provide referrals of one or service providers to clients for servicing one or more client needs. Client manager 112 searches the service provider database 116 for service providers that can service needs of a client and provides one or more service providers as referrals for the client. For example, when the client is determined as food insecure, client manager 112 searches the service provider database 116 and provides referrals of one or more service providers that can service the client's food insecurity. In one embodiment, client manager 112 may cross reference the client data 118 of the client with the service provider data stored in the service provider database 116 based on one or more client requirements. Client requirements may include personal preferences of the client including, but not limited to, proximity of the service provider location to a place of residence or place or employment of the client, preference of hours of operation of the service provider location and language preference. For example, client manager 112 may search the service provider database 116 for service providers that can service at least one need of the client, and additionally can satisfy client preferences including proximity to place of residence, longer hours of operation and can service the client in a preferred language. In an embodiment, client requirements may be manually entered and stored in memory 114, for example, as part of the client data 118. Additionally or alternatively, client manager 112 may cross reference client data of the client with the social provider data to check whether the client satisfies one or more registration criteria of each service provider that provides a service corresponding to the client need and is in accordance with client requirements. Client manager 112 returns only those service providers as referrals for which the client satisfies one or more registration criteria.

In one or more embodiments, when providing service provider recommendations, client manager 112 takes into account a capacity of the service providers to handle clients. The capacity to handle clients may include a total number of clients that a service provider can handle and/or a number of clients that the service provider can handle for each service provided by the service provider. Client manager 112 refers only those service providers which have capacity to handle additional clients for a client need. In one embodiment, the client manager 112 tracks capacity of a service provider based on previous referrals made to the service provider.

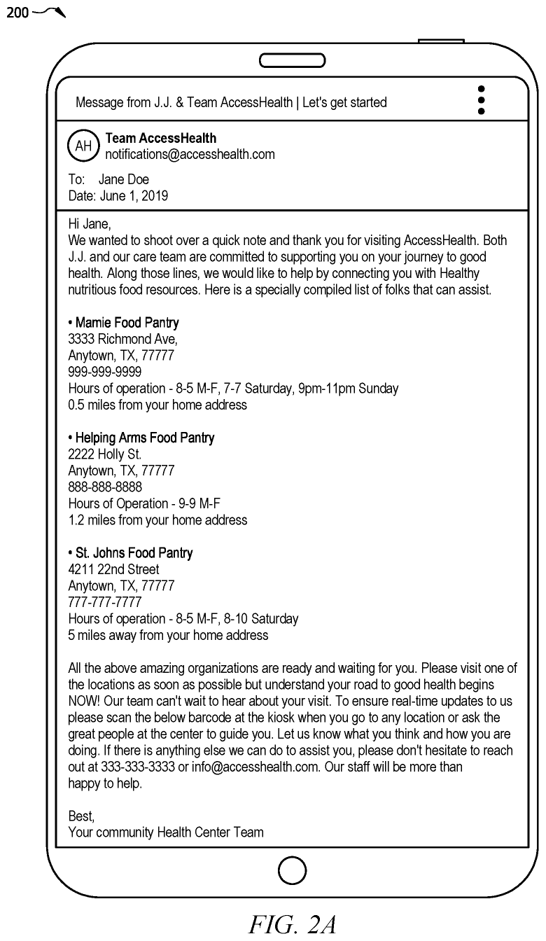

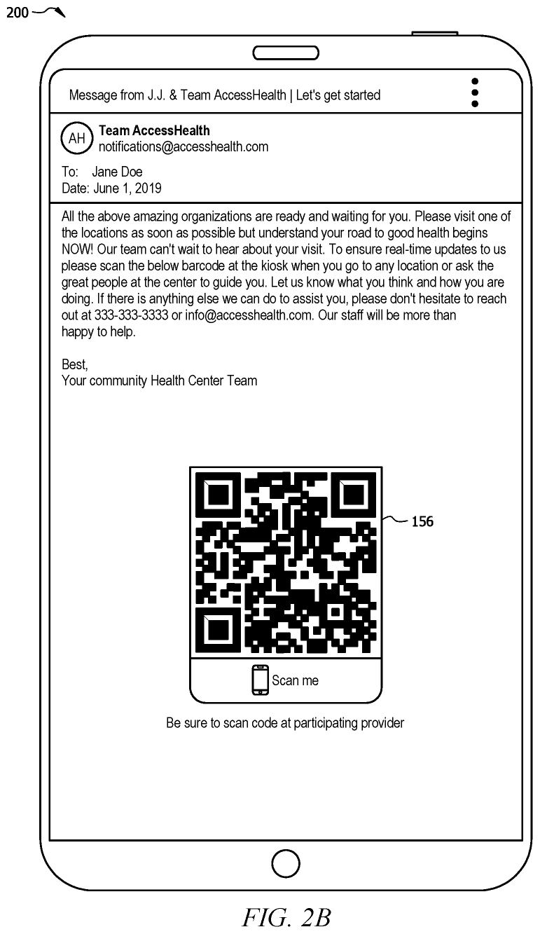

In one or more embodiments, once the service provider referrals are determined by the client manager 112, triage entity 110 may send a personalized notification to the client entity 140, wherein the notification includes information relating to the referred service providers. Client entity 140 may be any computing device the client owns or has access to and that can connect to network 170. For example, the client entity 140 may include a smart phone, tablet computer, laptop, desktop computer or the like. Triage entity 110 may send notifications to the client entity 140 via email, text message and/or any other known communication means. FIGS. 2A and 2B show an example email notification 200 sent out to a client including information relating to a number of service providers referred to service the client's food insecurity. As shown in FIG. 2A, the email notification may include a physical address of each service provider, contact details, hours of operation and distance from the client's home/work address. As shown in FIG. 2B, the example email notification additionally includes a unique bar code 156 which the client may scan using a mobile device 154 at a bar code scanner 152 provided at each of the referred service provider locations to indicate that the client has received services at the service provider location.

In one or more embodiments, triage entity 110 may send to each service provider referred to a client a portion of the client data 118 relating the client. Service provider entity 150 may be representative of a computing device belonging to a service provider referred to the client. The portion of the client data 118 sent to the service provider entity 150 may include, but is not limited to client identification information, contact information, client need to be serviced, and other information and documents that qualify the client to receive service from the service provider.

In one embodiment, service provider entity 150 may run a limited version of the client manager 112 (shown as 112a) with read-only access. In an alternative embodiment, the client manager 112a may be a thin client software application where the processing is directed by the thin client but largely performed by the triage entity 110 or a server hosting the client manager 112. A service provider using the client manager 112a running on the service provider entity 150 may access the portion of the client data 118 made available to the service provider.

As shown in FIG. 1 a barcode scanner 152 may be communicatively coupled to the service provider entity 150. A client may use the bar code scanner 152 to scan a unique bar code 156 provided to the client to indicate that the client has received services at the service provider location. In one embodiment, once a barcode is scanned at a referred service provided location, the service provider entity 150 sends out a notification to the triage entity 110 that the client has received services at the referred service provider location. Upon receiving the indication, the client manager 112 may record that the client has received the services.

In one or more embodiments, once the service provider referrals are determined for a client, client manager 112 may be configured to send client information and information relating to the service provider referrals to a care coordinator assigned to the client. The care coordinator may be a person assigned as a point of contact for the client and helps the client understand the process of receiving services from the referred service providers, keeps the client motivated to complete the process with each referred service provider and follow up with the client as needed. For example, client manager 112 may be configured to send at least a portion of the client data 118 and information including service provider referrals to care coordinator entity 180. Care coordinator entity 180 may be representative of a computing device belonging to a care coordinator assigned to the client. The portion of the client data 118 sent to the care coordinator entity 180 may include, but is not limited to client identification information, contact information, client need to be serviced, and information relating service provider referrals made to the client.

In one embodiment, care coordinator entity 180 may run a limited version of the client manager 112 (shown as 112b) with read-only access. In an alternative embodiment, the client manager 112a may be a thin client software application where the processing is directed by the thin client but largely performed by the triage entity 110 or a server hosting the client manager 112. A care coordinator using the client manager 112b running on the care coordinator entity 180 may access the portion of the client data 118 and other information made available to the care coordinator.

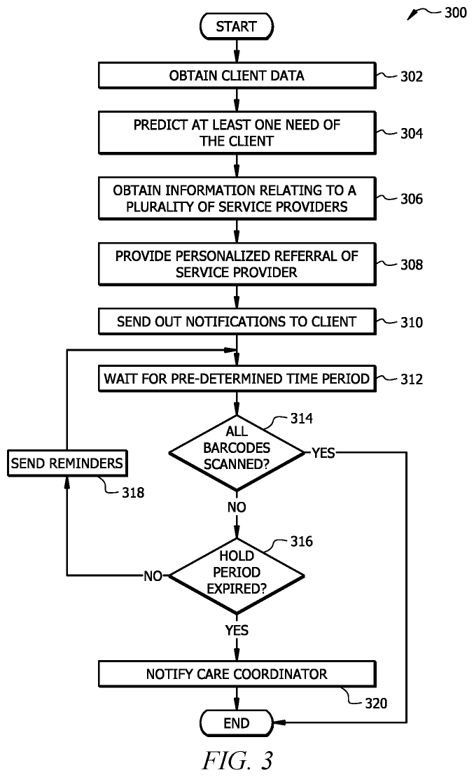

FIG. 3 is a flowchart of an example method 300 for managing social needs of a client, in accordance with certain embodiments of the present disclosure. The method 300 described herein may be performed by a triage entity 110 as described above with reference to FIG. 1.

At step 302, client manager 112 obtains client data 118 of the client stored in memory 114 of the triage entity 110. Client data 118 may include electronic health record (EHR) of the client. In one or more embodiments, the triage entity 110 may obtain EHR data of clients from EHR sub-system 130. EHR sub-system 130 may belong to any commercial EHR vendor providing EHR data. EHR of the client may include a range of data points including, but not limited to, demographics, medical history, medication and allergies, immunization status, laboratory test results, radiology images, vital signs, personal statistics like age and weight, and billing information. Client data 118 may also include information provided by the client to a health care provider managing the triage entity 110. For example, the information provided by the client may include, but is not limited to, age, gender, date of birth, social security number, income, home address, work address, weight, education level, employment status, military status, living situation and insurance status.

At step 304, client manager 112 predicts at least one need of the client based at least on the client data 118 of the client.

Client manager 112 may be configured to predict one or more needs of a client by identifying patterns in client data 118 relating to a plurality of clients. In one embodiment, client manager 112 is configured to identify correlation patterns 120 in client data 118, wherein each correlation pattern 120 correlates or maps one or more client data points with a client need. For example, a correlation pattern 120 may indicate that 90% of clients living in a particular zip code and having a household income less than $30,000 a year are food insecure. Following this example, for every client who lives in the same particular zip code and has a household income less than $30,000, client manager 112 may predict that the client is food insecure based on the correlation pattern 120.

Additionally or alternatively, client manager 112 may be configured to identify correlation patterns 120 in client data 118 based on demographic data 122 in order to predict client needs of clients. Such correlation patterns 120 based on demographic data may be referred to as demographic correlation patterns. For example, client manager 112 may have previously identified a data pattern based on client data 118 that 90% of clients living in a particular zip code have a household income less than $30,000 a year and are food insecure. Demographic data 122 may include data relating to other zip codes in the same city which have more than 90% of people living with household incomes less than $30,000 a year. Based on the previously identified data pattern in client data 118 and demographic data 122, client manager 112 may determine a demographic correlation pattern 120 which indicates that clients living in any zip code within the city and having household incomes lower than $30,000 a year are likely to be food insecure. Following this example, for every client who lives in any zip code of the city and has a household income less than $30,000, client manager 112 may predict that the client is food insecure based on the demographic correlation pattern 120.

In one or more embodiments, in order to identify a correlation pattern 120 in the client data 118, client manager 112 correlates client data points relating to a plurality of clients who have at least one common known need, and identifies common values of data points between the plurality of clients. For example, client manager 112 compares client data points of a plurality of clients known to be food insecure and identifies a subset of clients from the plurality of clients who live in the same zip code and have household incomes less than $30,000. In one embodiment, client manager 112 may designate an identified pattern in client data 118 as a correlation pattern 120 when more than a threshold number of clients having the same known need are identified to have the same one or more common data point values. For example, when client manager 112 identifies that more than 80% of clients living in the same particular zip code and having household incomes less than $30,000 are food insecure, client manager 112 designates this pattern as a correlation pattern 120 and stores the identified correlation pattern 120 in memory 114.

Once the correlation patterns 120 are identified and stored in memory 114, for every client, client manager 112 may correlate client data 118 of the client with each correlation pattern 120 stored in memory 114. When one or more data point values of the client data 118 of the client matches with corresponding one or more data point values of a correlation pattern 120, client manager 112 predicts that the client has a need associated with the correlation pattern 120. For example, when a correlation pattern 120 indicates that any client living in a particular zip code and having household income less than $30,000 is food insecure, the client living in the same particular zip code and having household incomes less than $30,000 is predicted to be food insecure.

In one or more embodiments, in order to identify a demographic correlation pattern 120, client manager 112 may correlate client data points in client data 118 relating to a plurality of clients who have at least one common known need, as well as to demographic data 122. Client manager 112 identifies common values of data points between the plurality of clients and compares the identified common data point values with demographic data 122, in order to identify demographic correlation patterns.

For example, client manager 112 may identify based on client data 118 that 90% of clients living in a particular zip code and having a household income less than $30,000 a year and are food insecure. Client manager 112 compares this correlation with demographic data 122 which may include data relating to other zip codes in the same city which have more than 90% of people with household incomes less than $30,000 a year. Based on the comparison of the previously identified correlation of client data point values in the client data 118 and additionally demographic data 122, client manager 112 may determine a demographic correlation pattern 120 which indicates that clients living in any zip code within the city having household incomes lower than $30,000 a year are likely to be food insecure.

Following this example, once the demographic correlation pattern 120 is identified and stored in memory 114, for every client, client manager 112 may correlate client data 118 of the client with the demographic correlation pattern 120 stored in memory 114. When one or more data point values of the client data 118 of the client matches with corresponding one or more data point values of the demographic correlation pattern 120, client manager 112 predicts that the client has a need associated with the demographic correlation pattern 120. For example, for the client who lives in any zip code of the city and has a household income less than $30,000, client manager 112 may predict that the client is food insecure based on the demographic correlation pattern 120.

At step 306, client manager 112 obtains information relating to a plurality of service providers, wherein each service provider of the plurality of service providers provides at least one service relating to a corresponding client need. Service provider database 116 may include information relating to a plurality of service providers providing an array of services to meet client needs. For example, information relating to each service provider may include several data points including, but is not limited to, legal name, tax identification (ID), general point of contact, full address (including city, state and zip code), office phone number, mobile phone number, email address, service location(s), service(s) provided and hours of operation, languages spoken, total capacity to handle clients, capacity for individual services, one or more registration criterion, documents required (e.g., insurance, proof of ID, address, military status etc.).

At step 308, client manager 112 provides personalized referral of a service provider to the client based at least one the client data 118, the predicted at least one need of the client and at least one registration criterion of the service provider.

In one or more embodiments, client manager 112 is configured to provide referrals of one or service providers to clients for servicing one or more client needs. Client manager 112 searches the service provider database 116 for service providers that can service needs of the client and provides one or more service providers as referrals for the client. For example, when the client is determined as food insecure, client manager 112 searches the service provider database 116 and provides referrals of one or more service providers that can service the client's food insecurity. In one embodiment, client manager 112 may cross reference the client data 118 of the client with the service provider data stored in the service provider database 116 based on one or more client requirements. Client requirements may include personal preferences of the client including, but not limited to, proximity of the service provider location to a place of residence or place or employment of the client, preference of hours of operation of the service provider location and language preference. For example, client manager 112 may search the service provider database 116 for service providers that can service at least one need of the client, and additionally can satisfy client preferences including proximity to place of residence, longer hours of operation and can service the client in a preferred language. In an embodiment, client requirements may be manually entered and stored in memory 114, for example, as part of the client data 118. Additionally or alternatively, client manager 112 may cross reference client data of the client with the social provider data to check whether the client satisfies one or more registration criteria of each service provider that provides a service corresponding to the client need and is in accordance with client requirements. Client manager 112 returns only those service providers as referrals for which the client satisfies one or more registration criteria.

In one or more embodiments, when providing service provider recommendations, client manager 112 may take into account a capacity of the service providers to handle clients. The capacity to handle clients may include a total number of clients that a service provider can handle and/or a number of clients that the service provider can handle for each service provided by the service provider. Client manager 112 refers only those service providers which have capacity to handle additional clients for a client need. In one embodiment, the client manager 112 tracks capacity of a service provider based on previous referrals made to the service provider.

At step 310, client manager sends out one or more notifications to the client with information relating the service provider referrals and other instructions relating to receiving services from the referred service providers.

In one or more embodiments, once the service provider referrals are determined by the client manager 112, triage entity 110 may send one or more personalized notifications to the client entity 140, wherein the notifications include information relating to the referred service providers. As discussed above, client entity 140 may be any computing device the client owns or has access to and that can connect to network 170. For example, the client entity 140 may include a smart phone, tablet computer, laptop, desktop computer or the like. Triage entity 110 may send notifications to the client entity 140 via email, text message and/or any other known communication means. Each notification may relate to a predicted client need and may include a physical address of each of the one or more service provider referred to service the client need, contact details, hours of operation and distance from the client's home/work address. Each notification may additionally include a unique bar code 156 which the client may scan using a mobile device 154 at a bar code scanner provided at a referred service provider location to indicate that the client has received services at the service provider location.

In one or more embodiments, triage entity 110 may optionally send to each service provider referred to the client a portion of the client data 118 relating the client. The portion of the client data 118 sent to the service provider may include, but is not limited to client identification information, contact information, client need to be serviced, and other information and documents that qualify the client to receive service from the service provider.

In one or more embodiments, once the service provider referrals are determined for the client, client manager 112 may optionally send client information and information relating to the service provider referrals to a care coordinator assigned to the client. The care coordinator may be a person assigned as a point of contact for the client and helps the client understand the process of receiving services from the referred service providers, keeps the client motivated to complete the process with each referred service provider and follow up with the client as needed. For example, client manager 112 may be configured to send at least a portion of the client data 118 and information including service provider referrals to care coordinator entity 180 accessible by the care coordinator. The portion of the client data 118 sent to the care coordinator entity 180 may include, but is not limited to client identification information, contact information, client need to be serviced, and information relating service provider referrals made to the client.

In an embodiment, when the client is predicted to have more than a threshold number of needs (e.g., higher than 3 needs), client manager 112 may require the care coordinator setup an initial meeting with the client to explain the process before triggering notifications to the client at step 310. For example, when the client has more than a given number of needs, the client may be overwhelmed by the amount of information the client receives (e.g., related to the service provider referrals) and the number of actions to be taken at one time. In such a case, talking to a care coordinator first may help the client understand the process, thus increasing the likelihood the client takes necessary actions to receive services from the referred service providers. In this context, client manager 112 may additionally provide the care coordinator entity 180 information relating to a number of needs predicted for the client and whether an initial meeting needs to be set up with the client. The care coordinator, upon completing the initial meeting with the client, may send a confirmation message to the triage entity 110 indicating that the initial meeting has been completed. In response, the triage entity 110 may send out the initial notifications to the client including information relating to service provider referrals and other information as described above.

At step 312, after sending out the initial notifications to the client, client manager 112 waits for a pre-determined time period without taking any further action. At step 314, once the pre-determined time period has expired, client manager checks whether all barcodes provided to the client have been scanned at respective service provider locations referred to the client. For example, when the client has three needs the client may be provide with three separate barcodes, one corresponding to each client need. If all barcodes have been scanned by the client, it means that the client has received series corresponding to all predicted client needs and the method 300 ends here.

However, when client manager 112 detects that the client has not scanned one or more barcodes provided to the client, it indicates that the client has not received services for corresponding one or more predicted client needs. In this case, method 300 proceeds to step 316, where client manager 112 checks whether a hold period has expired since sending initial notifications to the client at step 310. If the hold period has not expired, client manager 112 sends reminders to the clients corresponding to those client needs for which the bar codes have not been scanned at respective service provider locations.

In an embodiment, the hold period is selected to be longer than the pre-determined time period of step 312 to allow for multiple reminders to the client. Further, the pre-determined time period of step 312 is selected to be sufficiently long to allow the client time to set up appointments with service providers and receive services. For example, the pre-determined time period of step 312 may be set to 7 days and the hold period may be set to 25 days from sending initial notifications to the client. This allows for at least two reminders to the client before the hold period expires.

When client manager detects at step 316 that the hold period has expired, method 300 proceeds to step 320 where client manager 112 sends out a notification to the care coordinator (e.g., care coordinator entity 180) to follow up with the client and help the client take necessary steps to receive care from the referred service providers.

As may be appreciated from the above discussion, the care coordinator speaks to the client only when the client is a high need client and/or when the client is unable to receive services corresponding to certain client needs. This allows most clients to receive care without ever needing to talk to a care coordinator, thus significantly reducing the number of care coordinators needing to be employed by an organization to provide triage services to clients.

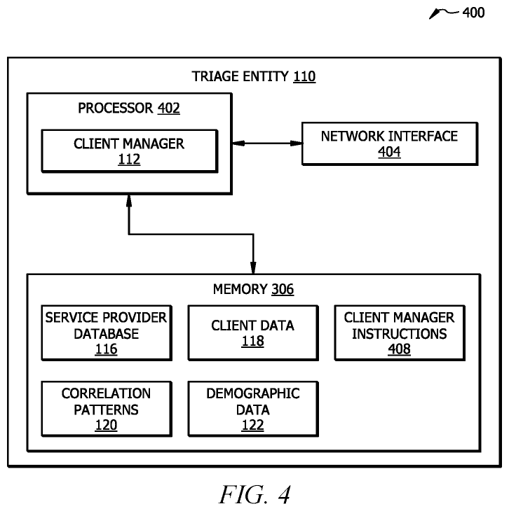

FIG. 4 illustrates an example schematic diagram of the triage entity 110, in accordance with one or more embodiments of the present disclosure.

The triage entity comprises a processor 402, a memory 114, and a network interface 404. The triage entity may be configured as shown in FIG. 4 or in any other suitable configuration.

The processor 402 comprises one or more processors operably coupled to the memory 114. The processor 402 is any electronic circuitry including, but not limited to, state machines, one or more central processing unit (CPU) chips, logic units, cores (e.g. a multi-core processor), field-programmable gate array (FPGAs), application specific integrated circuits (ASICs), or digital signal processors (DSPs). The processor 402 may be a programmable logic device, a microcontroller, a microprocessor, or any suitable combination of the preceding. The processor 402 is communicatively coupled to and in signal communication with the memory 114. The one or more processors are configured to process data and may be implemented in hardware or software. For example, the processor 402 may be 8-bit, 16-bit, 32-bit, 64-bit or of any other suitable architecture. The processor 402 may include an arithmetic logic unit (ALU) for performing arithmetic and logic operations, processor registers that supply operands to the ALU and store the results of ALU operations, and a control unit that fetches instructions from memory and executes them by directing the coordinated operations of the ALU, registers and other components.

The one or more processors are configured to implement various instructions. For example, the one or more processors are configured to execute instructions (client manager instructions 408) to implement the client manager 112. In this way, processor 402 may be a special-purpose computer designed to implement the functions disclosed herein. In one or more embodiments, the client manager 112 is implemented using logic units, FPGAs, ASICs, DSPs, or any other suitable hardware. The client manager 112 is configured to operate as described with reference to FIGS. 1-3. For example, the client manager 112 may be configured to perform at least a portion of the flowchart 300 as described in FIG. 3.

The memory 114 comprises one or more disks, tape drives, or solid-state drives, and may be used as an over-flow data storage device, to store programs when such programs are selected for execution, and to store instructions and data that are read during program execution. The memory 114 may be volatile or non-volatile and may comprise a read-only memory (ROM), random-access memory (RAM), ternary content-addressable memory (TCAM), dynamic random-access memory (DRAM), and static random-access memory (SRAM).

The memory 114 is operable to store the client manager instructions 408 and/or any other data or instructions. The client manager instructions 408 may include any suitable set of instructions, logic, rules, or code operable to execute the client manager 112.

The network interface 404 is configured to enable wired and/or wireless communications. The network interface 404 is configured to communicate data between the triage entity 110 and other devices (e.g. EHR sub-system 130, client entity 140, service provider entity 150 and care coordinator entity 180), systems, or domains.

For example, the network interface 404 may comprise a Wi-Fi interface, a LAN interface, a WAN interface, a modem, a switch, or a router. The processor 402 is configured to send and receive data using the network interface 404. The network interface 404 may be configured to use any suitable type of communication protocol as would be appreciated by one of ordinary skill in the art.

It may be noted that each of the EHR system 130, client entity 140 service provider entity 150 and care coordinator entity 180 may be implemented similar to the triage entity 110 as shown in FIG. 1. For example, each of the EHR system 130, client entity 140 service provider entity 150 and care coordinator entity 180 may include a processor and a memory storing instructions to implement the respective functionality of the entity when executed by the processor.

While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise. Other examples of changes, substitutions, and alterations are ascertainable by one skilled in the art and could be made without departing from the spirit and scope disclosed herein.

To aid the Patent Office, and any readers of any patent issued on this application in interpreting the claims appended hereto, applicants note that they do not intend any of the appended claims to invoke 35 U.S.C. § 112(f) as it exists on the date of filing hereof unless the words “means for” or “step for” are explicitly used in the particular claim.

1. A system comprising:
at least one processor configured to:
obtain client data of a client;
predict at least one need of the client based at least on the client data;
obtain information relating to a plurality of service providers, wherein each service provider provides at least one service relating to a corresponding need; and
provide personalized referral of a service provider to the client, based at least on the client data and the predicted at least one need of the client.

2. The system of claim 1, wherein the client data comprises one or more of data from an Electronic Health Record (EHR) of the client and other information obtained from the client.

3. The system of claim 1, wherein the at least one processor is further configured to:
correlate the client data of a plurality of clients having at least one known need common to the plurality of clients; and
determine one or more correlation patterns based on the correlation, wherein each correlation pattern maps the at least one known need to one or more common data points of the client data relating to the plurality of clients.

4. The system of claim 3, wherein correlating the client data of the plurality of clients comprises correlating one or more data points of the client data relating to a first client of the plurality of clients to corresponding one or more data points of the client data relating to a second client of the plurality of clients, wherein the first client and the second client have the same at least one known need.

5. The system of claim 1, wherein the at least one processor predicts the at least one need of the client by:
comparing one or more data points of the client data of the client with each of a plurality of predetermined correlation patterns, wherein each correlation pattern indicates a need based on one or more data points of the client data;
determining that at least one data point of the one or more data points matches with corresponding at least one data point of a predetermined correlation pattern; and
predicting one or more needs of the client corresponding to the predetermined correlation pattern.

6. The system of claim 1, wherein the at least one processor predicts the at least one need of the client by:
correlating client data with demographic data related to the client;
determining that the correlation matches with a predetermined demographic pattern of correlation, wherein the demographic pattern maps at least one need to one or more data points of the client data; and
predicting the at least one need of the client corresponding to the demographic pattern.

7. The system of claim 1, wherein the at least one processor provides personalized referral of the service provider by:
determining at least one service provider that can service the need of the client and satisfy one or more client requirements, wherein the one or more client requirements include at least one of proximity of a service location of the at least one service provider to at least one of a residential address or a work address of the client or suitable hours of operations.

8. The system of claim 1, wherein the at least one processor provides the personalized referral of the service provider by:
determining, based on the client data, whether the client satisfies at least one registration criterion of the service provider.

9. The system of claim 1, wherein the at least one processor is further configured to:
transmit information related to the referral to the referred service provider, wherein the information related to the referral comprises one or more of at least a portion of the client data of the client, information relating to the need of the client and copies of one or more documents needed to establish that the client satisfies the registration criterion of the service provider

10. The system of claim 1, wherein the at least one processor is further configured to:
receive an indication that the client has received a service relating to the predicted need from the service provider; and
record that the client has received the service from the service provider.

11. The system of claim 1, wherein the at least one need of the client comprises one or more of food, housing, clothing, utilities, transportation, legal needs, recreation, employment and education.

12. A method for managing needs of a client, comprising:
obtaining client data of a client;
predicting at least one need of the client based at least on the client data;
obtaining information relating to a plurality of service providers, wherein each service provider provides at least one service relating to a corresponding need; and
providing personalized referral of a service provider to the client, based at least on the client data and the predicted at least one need of the client.

13. The method of claim 12, further comprising:
correlating the client data of a plurality of clients having at least one known need common to the plurality of clients; and
determining one or more correlation patterns based on the correlation, wherein each correlation pattern maps the at least one known need to one or more common data points of the client data relating to the plurality of clients.

14. The method of claim 12, wherein predicting the at least one need of the client comprises:
comparing one or more data points of the client data of the client with each of a plurality of predetermined correlation patterns, wherein each correlation pattern indicates a need based on one or more data points of the client data;
determining that at least one data point of the one or more data points matches with corresponding at least one data point of a predetermined correlation pattern; and
predicting one or more needs of the client corresponding to the predetermined correlation pattern.

15. The method of claim 12, wherein providing personalized referral of the service provider comprises:
determining at least one service provider from a service provider database that can service the need of the client and satisfy one or more client requirements, wherein the one or more client requirements include at least one of proximity of a service location of the at least one service provider to at least one of a residential address or a work address of the client or suitable hours of operations.

16. The method of claim 12, further comprising:
receiving an indication that the client has received a service relating to the predicted need from the service provider; and
recording that the client has received the service from the service provider.

17. A computer-readable medium storing instructions which when executed by a processor perform a method for managing needs of a client, the method comprising:
obtaining client data of a client;
predicting at least one need of the client based at least on the client data;
obtaining information relating to a plurality of service providers, wherein each service provider provides at least one service relating to a corresponding need; and
providing personalized referral of a service provider to the client, based at least on the client data and the predicted at least one need of the client.

18. The computer-readable medium of claim 17, further comprising instructions for:
correlating the client data of a plurality of clients having at least one known need common to the plurality of clients; and
determining one or more correlation patterns based on the correlation, wherein each correlation pattern maps the at least one known need to one or more common data points of the client data relating to the plurality of clients.

19. The computer-readable medium of claim 17, wherein predicting the at least one need of the client comprises:
comparing one or more data points of the client data of the client with each of a plurality of predetermined correlation patterns, wherein each correlation pattern indicates a need based on one or more data points of the client data;
determining that at least one data point of the one or more data points matches with corresponding at least one data point of a predetermined correlation pattern; and
predicting one or more needs of the client corresponding to the predetermined correlation pattern.

20. The computer-readable medium of claim 17, wherein providing personalized referral of the service provider comprises:
determining at least one service provider from a service provider database that can service the need of the client and satisfy one or more client requirements, wherein the one or more client requirements include at least one of proximity of a service location of the at least one service provider to at least one of a residential address or a work address of the client or suitable hours of operations.
